(19) United States
(12) Patent Application Publication
(10) Pub. No.: US 2022/0172806 A1
(43) Pub. Date: Jun. 2, 2022

(54) ELECTRONIC HEALTH RECORD SYSTEM

(71) Applicant: Orangehook, Inc., Wayzata, MN (US)

(72) Inventors: Ian Worden, Gilbert, AZ (US); Noah M. Coad, Cataldo, ID (US)

(21) Appl. No.: 17/564,896

(22) Filed: Dec. 29, 2021

Related U.S. Application Data
(63) Continuation of application No. 14/286,912, filed on May 23, 2014, now abandoned.
(60) Provisional application No. 61/826,996, filed on May 23, 2013.

(51) Int. Cl. G16H 10/60

(52) U.S. Cl. CPC G16H 10/60

(57) ABSTRACT

A system and method provides central storage of patient health records and allows for temporal access to be provided by the patient to other individual and healthcare providers.

### CROSS REFERENCE TO RELATED APPLICATION

The present application is a continuation of U.S. Nonprovisional application Ser. No. 14/286,912, May 23, 2014, which claims the benefit of U.S. Provisional Application Ser. No. 61/826,996, filed May 23, 2013, which application is incorporated herein by reference in its entirety.

### FIELD OF THE DISCLOSURE

The present disclosure is related computer-implemented systems and methods for storing and providing access to health records and recording and storing consent for the same.

### DESCRIPTION OF THE BACKGROUND ART

One of the reasons that healthcare costs continue to rise is the lack of a central health record for a patient. Each healthcare provider stores a copy of the patient's record with that office and thus the patient's record is spread across multiple healthcare providers. This is specifically a problem where various healthcare providers are not all part of one system. In order for a healthcare provider to obtain a copy of a patient's record from a second healthcare provider, the patient provides a one-time consent that is sent to the second healthcare provider who then faxes or mails a copy of the requested file to the requesting healthcare provider. Additional tools are needed to allow for consolidation of a patient's medical records and streamlining the process for a patient to provide consent to a healthcare provider to have access to all of the patient's records.

### SUMMARY

A system and method for stores health records and provides temporal access to the record at the request of the patient. Upon receiving authentication information from a patient, the health record system provides a designated recipient, such as a healthcare provider, access to the patient's health record. In some embodiments, two pieces of authentication information are received from the patient and a third piece of authentication information is received from the designated recipient. In some embodiments, a first patient can authorize the designated recipient to not only view the health record but also provide access to the health record.

### BRIEF DESCRIPTION OF THE DRAWINGS

FIG. 1 is a diagram of system architecture according to one embodiment.

FIG. 2 is a data flow chart showing a method for a patient to provide consent for a healthcare provider to access remotely stored healthcare records according to one embodiment.

FIG. 3 is an alternative view of the use of the health record system 100.

FIGS. 4A and B illustrate example user interfaces presented on the client at the provider's office before and after insertion of the card into the reader.

FIGS. 5A and B illustrate an example card.

FIG. 6 illustrates an example user interface displaying insurance information.

FIG. 7 illustrates an example user interface for providing a pin to access health record information.

FIG. 8 illustrates an example user interface allowing a user to select a health record to display.

FIG. 9 illustrates an exemplary display of a health record.

FIG. 10 illustrates an example user interface for selecting fields to be included in a printed health record.

FIG. 11 illustrates an example user interface displaying a list of healthcare providers who have accessed the health record.

FIG. 12 illustrates a data flow chart of providing a first user consent to allow access to a second user's health record.

FIG. 13 illustrates an example user interface for a user to request to add another patient's health record to the user's card.

FIG. 14 illustrates an example user interface indicating that permission has been requested to a user to add that user's health record to the first user's card.

FIG. 15 illustrates an example user interface displaying to a user that permission has been requested to add that user's health record to another user's card.

FIG. 16 illustrates an example communication displaying to a user that permission has been requested to add that user's health record to another user's card.

The figures show various embodiments of the present invention for purposes of illustration only. One skilled in the art will recognize from the following description that alternative embodiments of the structures and methods shown may be used without departing from the principles of this invention.

### DETAILED DESCRIPTION

### Introduction

A system method for providing access to a health record associated with a patient to a health care provider comprises receiving a first and a second piece of authentication information from the patient; receiving a third piece of authentication information from the health care provider; and providing the health record associated with the patient to the health care provider.

A system and method for storing consent for a health care provider to access a health record associated with a patient comprises receiving an indication from a patient of consent for the health care provider to access the health record; providing the health record to the health care provider; and storing an indication of the health care provider's access to the health record as associated with the patient.

A system and method for a receiving and storing a first patient's consent to a second patient to access to a health record associated with the first patients, the method comprises receiving from the first patient consent for the second patient to access the health record associated with the first patient; storing the received consent as associated with the second patient; receiving a request from the second patient to provide the health record associated with the first patient to a health care provider; and providing the health record associated with first patient to the health care provider.

### System Overview

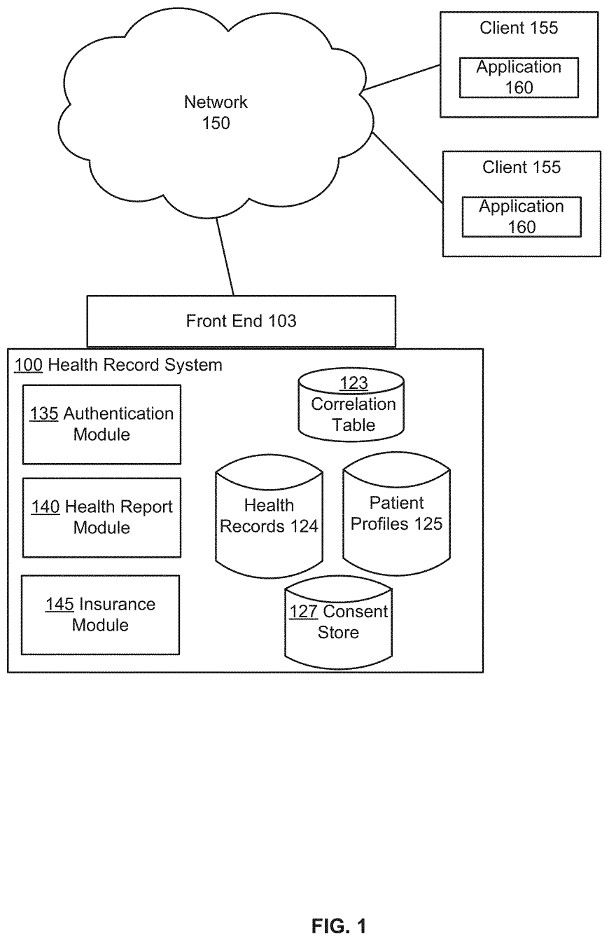

FIG. 1 is a diagram of system architecture according to one embodiment. The health record system 100 comprises an authentication module 135, a health report module 140, an insurance module 145, a correlation table 123, health records 124 and patient profiles 125. For simplicity only one health record system 100, authentication module 135, health report module 140, insurance module 145, correlation table 123, health records 124 and patient profiles 125 are shown, but in practice many of each may be in operation. The health records 124 may be located remotely from the health record system 100 and be accessed via the network 150.

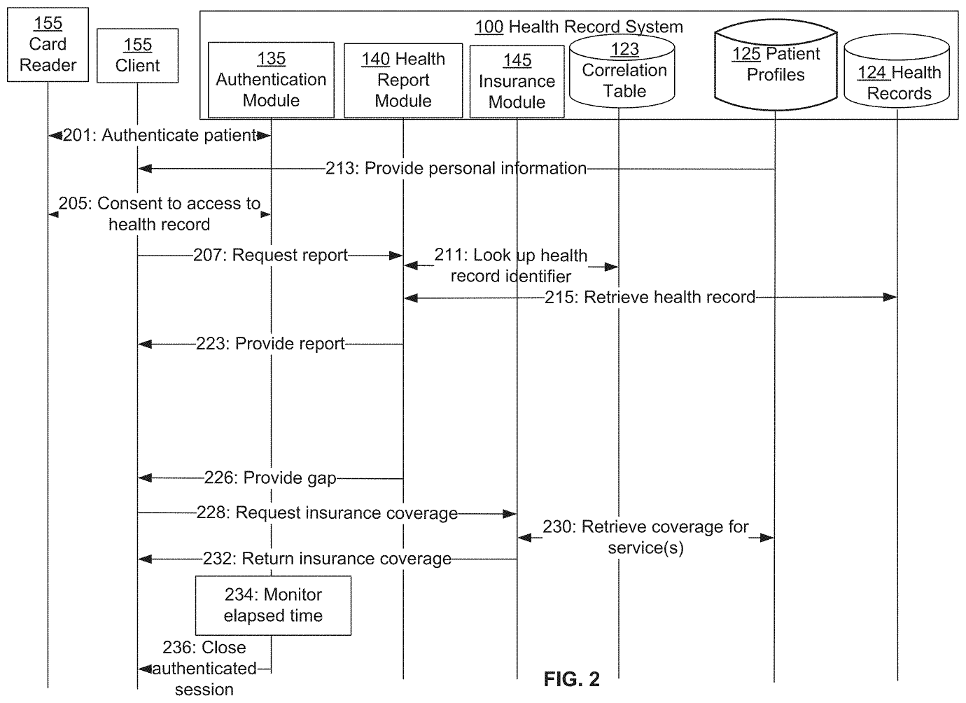

The health record system 100 provides patients and health care providers access to health insurance information (e.g., coverage, limits, requirements, co-payments) as well as health records of the patients and is one means for performing this function. Patients and health care providers authenticate to the health record system 100 using authenticating data received from a client device 155. The authentication process will be discussed in greater detail in reference to FIG. 2. The health record system 100 communicates with one or more clients 155 via a network 150 and the front end 103. The network 150 is typically the Internet, but may also be any network, including but not limited to a LAN, a MAN, a WAN, a mobile, wired or wireless network, telecommunication network, a private network, or a virtual private network, and any combination thereof.

The client 155 is any type of device that is adapted to access the health record system 100 over the network 150. Examples of clients 155 include, but are not limited to, mobile devices such as a handheld computer, laptop computer, desktop computer, tablet computer, mobile phone or personal digital assistant (PDA). Most basically, a client 155 is configured to transmit information authenticating information to the health record system 100 and receiving health record information. In an exemplary embodiment, a provider's office has a client 155 at which the provider views the patient's health record as well as a client 155 that is capable of reading the patient's card and receiving authentication information input by the patient. This can be a card reader with a key pad and/or touch screen. A client 155 also allows the patient to update his or her personal information and health record. The client 155 further comprises a client application 160 for requesting authentication of providers and patients to the health record system 100 and display health records after authentication. Applications 160 at patient client devices 155 allow patients to update health records and personal information. For simplicity only two clients 155 are shown. In practice, very large numbers (e.g., millions) of clients 155, or as many as can be supported by the hardware and software implementation, can be in communication with the health record system 100 at any time.

The health record system 100 is a computer system implemented as server program executing on one or more server-class computers comprising a CPU, memory, network interface, peripheral interfaces, and other well-known components. The computers themselves preferably run an open-source operating system such as LINUX, have generally high performance CPUs, with 1 G or more of memory, and 1 Tb or more of disk storage. Of course, other types of computers can be used, and it is expected that as more powerful computers are developed in the future, they can be configured in accordance with the teachings here. The functionality implemented by any of the elements can be provided from computer program products that are stored in non-transitory tangible computer accessible storage mediums (e.g., RAM, hard disk, or optical/magnetic media), or by equivalent implementations in hardware and/or firmware. As will become apparent from the description below, the operations of the health record system 100, and in particular, the management of health records and provision thereof to authorized entities only are sufficiently complex as to require their implementation in a computer system, and cannot be performed in the human mind by mental steps alone.

The authentication module 135 processes the authentication requests from client devices 155 of patients and health care providers to the system and is one means for performing this function. The authentication module 135 receives authenticating information such as user names, passwords and PINS.

The patient profiles 125 stores personal information about patients other than health information and is one means for performing this function. For example, a patient's profile includes name, date of birth, address, insurance coverage(s) including patient's policy or membership identification number for each. Patient profile data are received from the patient when the patient enrolls as well and additionally when the patient activates the means for authenticating to the health record system 100. The health record system 100 receives updates about a patient's insurance coverage (e.g., whether insurance remains in effect and levels of coverage) from the insurance company which is then stored as associated with that patient in patient profiles 125. In some embodiments, updates to insurance information are received daily, weekly or monthly. The updates to the information can be in the form of exception reports. This keeps the amount of data received low and identifies just what has changed since the last update.

The health records 124 stores a patient's health records and is one means for performing this function. Health records include records of physician office visits, test results, hospitalizations, allergies, immunizations, current and past medications, diagnoses, etc. Health records for a patient are obtained from a variety of sources including the patient, the patient's healthcare providers and from insurance claims processed for the patient. The health records information is standardized if needed. Information received from insurance companies and doctors' offices is usually received in a standardized form such as, for example, the International Statistical Classification of Diseases and Related Health Problems (ICD-9) codes for diagnoses and Current Procedural Terminology (CPT) codes for procedures. As new formats for data are created (e.g., ICD-10), they can be used with the disclosed system. Patients input history via menu selections such that each item is associated with standardized identifications. Some information may be provided in free text. The creation and maintenance of a patient's health record is discussed further below. In some embodiments, the health records are de-identified. In some embodiments, de-identified records are the health record without any personally identifiable information such as the patient's name, birth date, etc. In some embodiments, the records are de-identified as outlined in 45 CFR § 164.514(b). This provides security in case of unauthorized access to health records 124. In an embodiment where health records are de-identified a correlation table 123 stores the identifier for each patient's health record in health records 124. In some embodiments, the correlation table 123 is stored remotely from the health record system 100.

Health records 124 further stores information about gaps in health care for each patient. A gap in health care is a recommended procedure or test that is missing from the patient's health record. For example, tetanus shots are recommended every 10 years. Mammograms are recommended at regular intervals for women above a threshold age. Gaps in care are received by the health record system 100 from a variety of outside sources, such as the patient's insurance company, and stored in health records 124. In other embodiments, the health record system 100 analyzes health records 124 and determines gaps in care, using various rules based on age, gender, health status, and healthcare insurance requirements, and clinical best practices. For each identified gap, an indication of the specific type of gap (e.g., tetanus immunization), along with additional information (e.g., date the procedure or test was due) is stored in the patient's health record 124.

The health report module 140 retrieves health reports to be provided to the client 155 and is one means for performing this function. Additionally, the health report module 140 processes received insurance claims for a patient into health report information. In one embodiment, processing the insurance claims comprises identifying individually billed procedures for a patient that all relate to a single event into a single diagnosis or procedure in the patient's health report. For example, if a patient has a deep laceration on a finger, the procedures can include an x-ray, stitches and a consultation with a physician. The patient may need a tetanus booster and antibiotics might be prescribed. Each of these procedures will appear separately on the bill(s) to the insurance company, but it would unnecessarily increase the size of the patient's health record to include every individual detail of the related procedures for this event. Thus, the health report module 140 groups all of the line items into an entry in the health report reading “Deep laceration to right index finger” and the date. From insurance claims for prescription medications, the health report module 140 populates the portion of the health record with the name and dosage of medications being taken by the patient. Attached as Appendix A is an example of a complete health record.

The consent store 127 stores the consents that are associated with each patient's authentication information. A consent is ability to access a health record. In some embodiments, a patient's authentication information is associated with consents for multiple health records—the patient's own as well as the health record of the patient's children and/or other individuals who have granted the patient the right to access their health records. The consent allows the patient to access his or her own health record but also allows the patient to enable a healthcare provider to access the health record. In some embodiments, the authentication information is a PIN. In some embodiments, a consent is created when the patient creates an account including authentication information with the health record system 100. The health record system 100 provides terms and conditions for display and review by the patient. These terms and conditions describe how the health record system 100 provides access to the patient's health records when the patient's authentication information us received at the health record system 100. Upon receipt at the health record system 100 indication of the patient's agreement to those terms and conditions (e.g., by click, electronic signature or a hard copy signature), the consent is stored in the consent store 127.

### Process Overview—Accessing Health Records

The operation of the health record system 100 is described in reference to FIGS. 2-10. Patients have portable card the size of a credit card that stores information. Example information stores include an integrated circuit (“chip”) or magnetic strip. The cards that have been activated after receipt by the patient. The activation process includes verification that the person activating the card is in fact the patient.

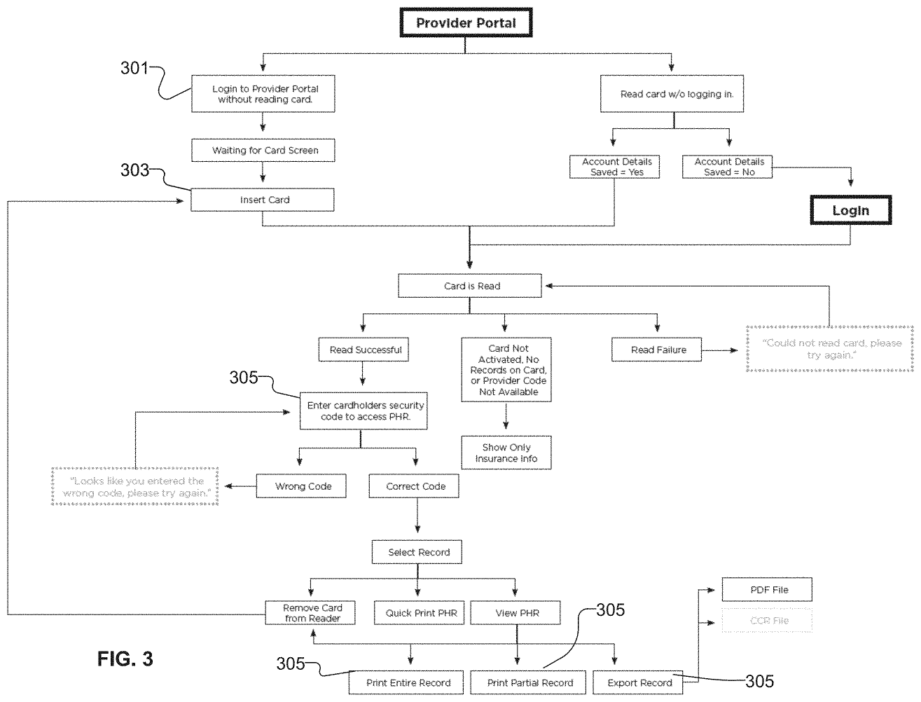

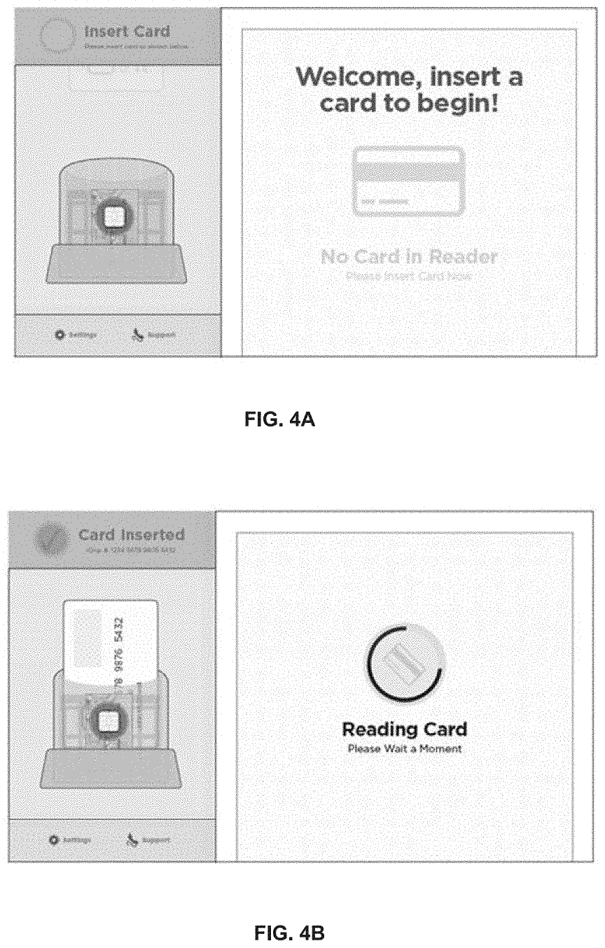

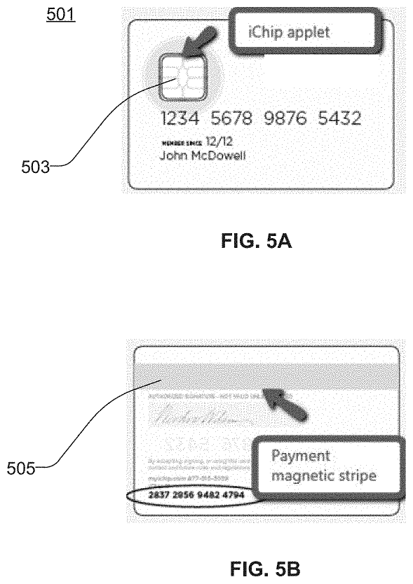

FIG. 2 is a sequence diagram illustrating the use of the health record system 100 according to one embodiment. FIG. 3 is an alternative view of the use of the health record system 100. Referring to FIG. 2, upon arrival at a doctor's office or pharmacy or other health service provider, the patient authenticates 201 to the health record system 100 with the patient's authentication information. The user at the health service provider, such as the receptionist, also logs in to the health record system, also at a client 155, providing the provider account's authentication information, such as a secure user name and password, and any additional authentication information required by the system 100. In one embodiment, a card issued to the patient is inserted into, or otherwise read by, a compatible card reader client 155 at the provider's location. FIG. 4A is an example user interface presented on the card reader client 155 at the provider's office. After the card has been inserted into the card reader client 155, FIG. 4B illustrates the initial reading of the card 401 by the card reader client 155. The reader reads the information on the card either by reading a magnetic strip, an integrated circuit (“chip”) 403 or via wireless communication such as RFID or near field communication (NFC). The card includes authentication information for the card holder, such as a profile number. The card reader client 155 transmits the authentication information as well as information identifying the provider to the health record system 100, which authenticates the card and provides to provider client 155 basic personal information about the patient. The authentication process uses, for example, a public/private key system. The basic personal information can also be displayed at the card reader client 155. FIG. 5 illustrates an example card having both a chip 403 and magnetic strip 505. The card may also be a payment device in the form of a debit card, credit card or prepaid card. The payment functionality can be either in the chip 403 or the magnetic strip 505 or both.

In another embodiment, the patient hands the card to the receptionist and the card is read by a client 155 used by the provider. The patient then also enters the authentication information including PIN for access to the health record at that provider client 155. Such an embodiment is described at FIG. 3. The user at the provider logs in 301. The card is inserted 303 in a reader. Authentication information stored on the card is transmitted to the health record system 100 and the basic information about the patient is displayed to the provider at client 155. The patient provides 305 second authentication information such as a PIN. This is transmitted to the health record system 100 and responsive to the PIN being verified, the health record is provided to the client 155.

In another embodiment, no card is required and the patient is authenticated via an application on the patient's mobile device. Mobile devices include but are not limited to mobile phones, laptops, and tablet computers. The patient installs a client application 160 of the health record system 100 on their mobile device client 155, during which installation the identity of the patient is verified between the client application and the health record system 100. Then upon arriving at a provider office, the service provider's client 155 displays a Quick Response (QR), which the patient scans with the application on their mobile device client 155. The QR code identifies the provider office and account with the health record system 100. The mobile device client 155 communicates data from the QR code to the health record system 100 to authenticate the patient as being physically present at the provider office. Alternatively the mobile device client 155 receives the identification of the provider office and account with the health record system 100 via other means such as wireless communication such as NFC.

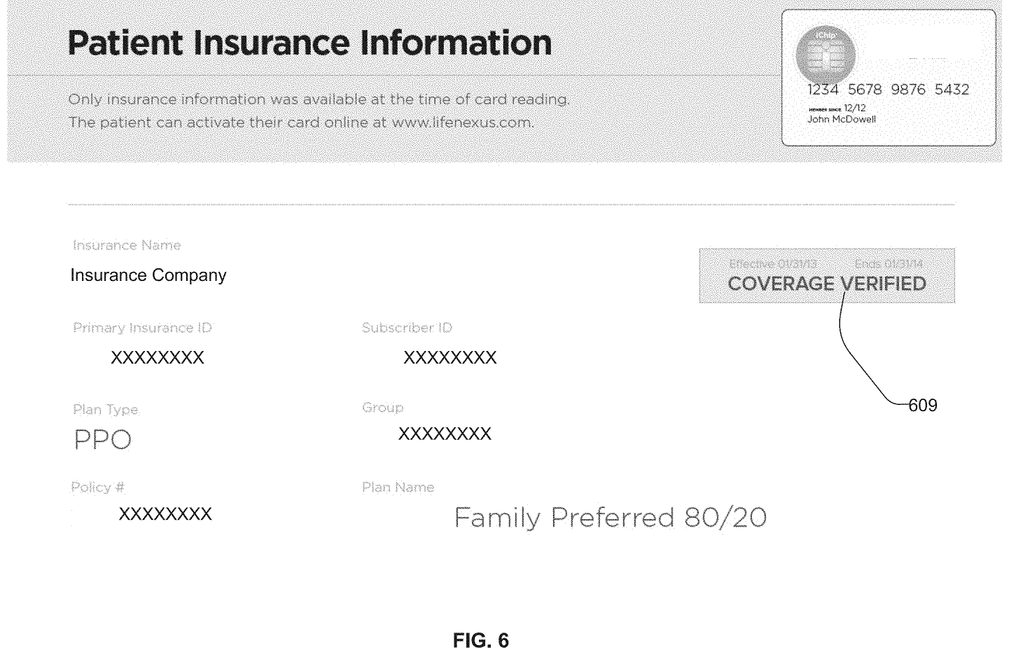

The basic personal information about the patient provided by the health record system 100 to the provider client 155 is from personal information 125 and includes the patient's identifying information and insurance information. The provider however does not yet have authority to view the patient's health records. FIG. 6 illustrates the user interface at provider client 155 displaying just insurance information. The information includes whether the patient's insurance is currently in effect 609.

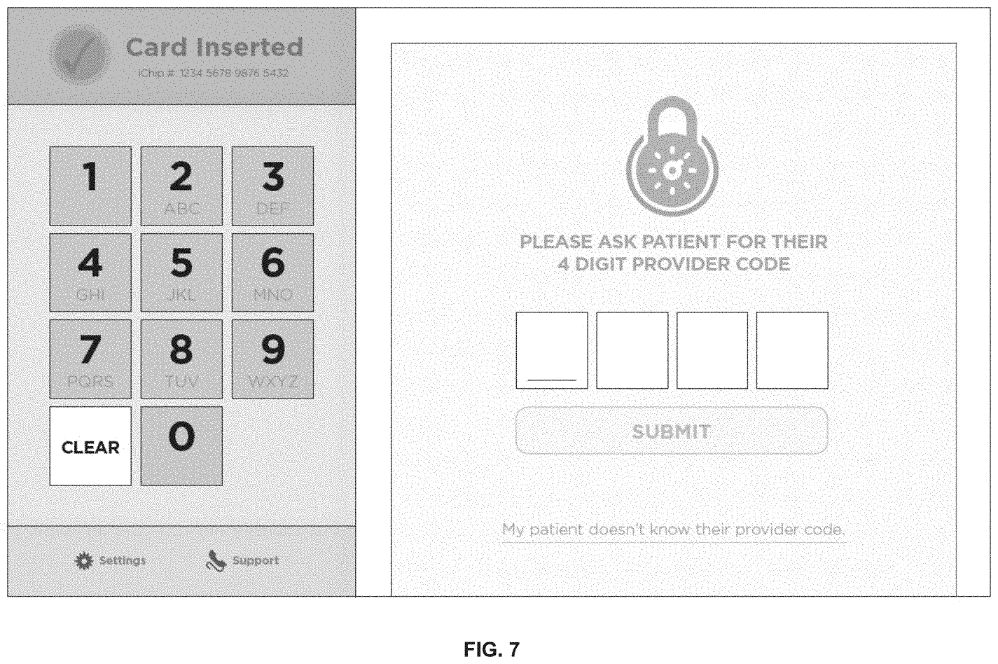

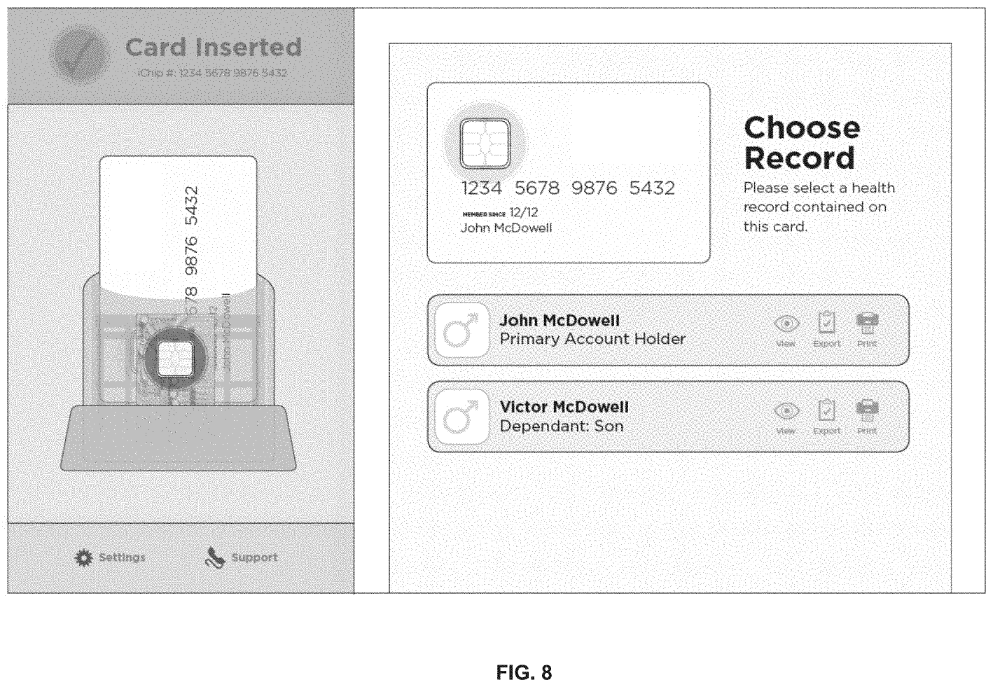

In order to allow the provider to receive the patient's health record, the patient consents 205 to the provider's access to the health record by providing a PIN which is input at the user interface of the client 155 as shown in FIG. 7. In an embodiment using a mobile device rather than a card, the patient inputs the PIN on the patient's mobile device to provide consent. As shown in FIG. 8, after verification of the PIN, the user interface displays the one or more health records associated with the card and available from the health record system 100. Thus, in this embodiment, access to a patient's health record requires multiple factors of authentication from multiple parties. The provider's office must be logged in to the health record system 100 using a user name and password. The patient authenticates him or herself using either a card or a mobile application and provides consent for the provider to access the health record through presentation of a PIN. If any of these factors are missing, the patient's health record cannot be accessed by the provider office. Each time a patient consents for a provider to access to the patient's health record, that consent is recorded in the patient's profile in patient profiles 125. Optionally, the consent is also stored as associated with the health record itself in health records 124.

In embodiments where more than one health record is associated with the card, patients may be able to set up multiple PINs—one for each health record associated with the card and optionally one master PIN for all health records so that the provider only has access to one health record. For example, as shown in FIG. 8, the card is associated with the health records of John McDowell and his son, Victor McDowell. If each health record has its own PIN, John McDowell can select the PIN for his son's record when visiting the son's pediatrician. That way John McDowell's own health record is not available to the pediatrician. Conversely, when visiting his otolaryngologist, John McDowell can provide the PIN for his own health record so that his son's record is not available to the otolaryngologist's office.

Returning to FIG. 2, after the provider selects a health record to view, the health report module 140 receives a request 207 for the selected patient's health report. In an embodiment where the health record information is de-identified, the health report module 140 looks 211 up the patient's health record identifier in a correlation table 123. Using the retrieved identifier, the health report module 140 retrieves 215 the patient's health record from the health records 124 and provides 223 it for display at the provider client device 155. If any gaps in care are indicated in the patient's health record, the gap information is provided 226 as well.

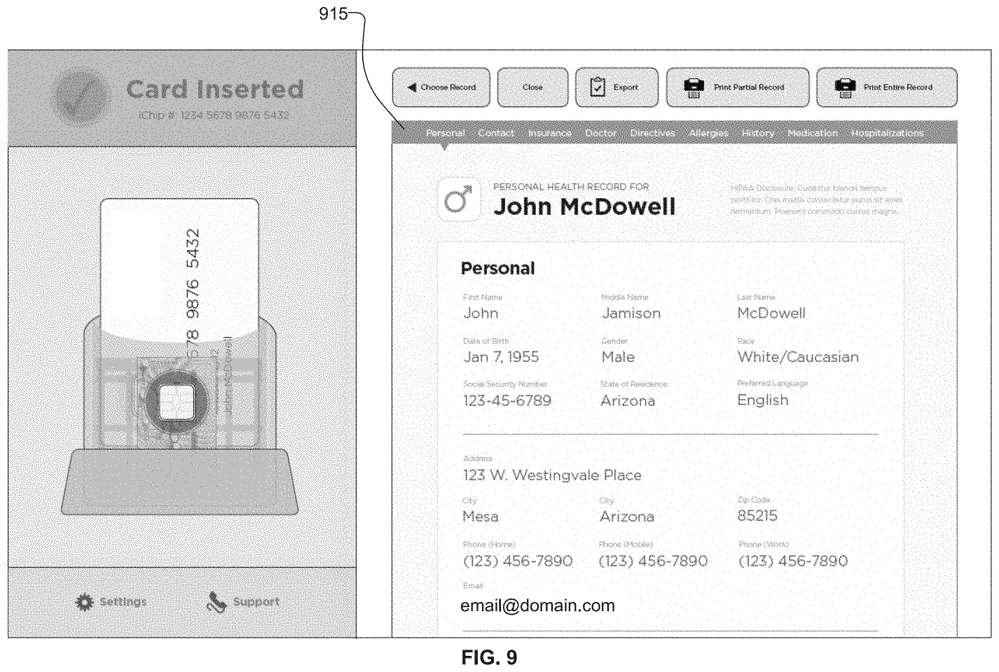

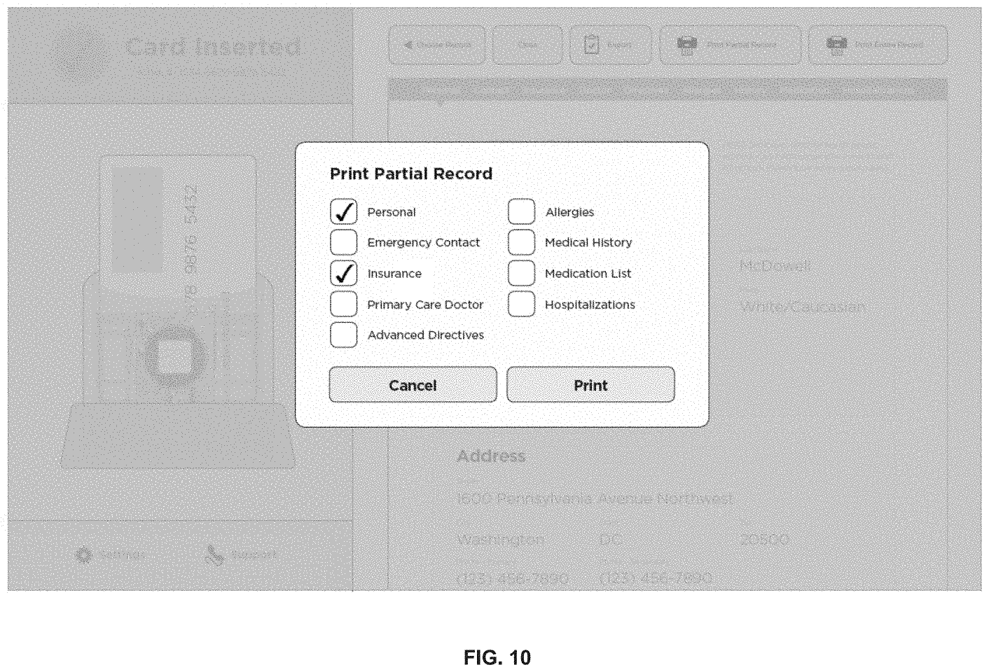

The health record for John McDowell, is displayed as shown in FIG. 9. Links on bar 915 provide access to the various portions of John McDowell's health record—Personal, Contact, Insurance, Doctor, Directives, Allergies, History, Medication and Hospitalizations. Buttons above bar 915 provide functions like exporting and printing all or some of the health record. These features are also shown at 307 of FIG. 3. If selecting to print only a partial record, the provider selects the categories of items that are to be included in the printed document as shown in FIG. 10.

The user of the service provider's client 155 can also request 228 insurance coverage for a particular procedure from the insurance module 145 which retrieves the information from patient profiles 125 and returns 232 the information to client 155.

The authentication of the patient to the client device 155 at the provider, along with access to one or more health records, can be shared by the client device 155 with other client devices 155 connected to the first client device 155 via a network. For example, authentication of the patient and granting of access to the health records takes place at a client device 155 in the reception area of a provider and then additional client devices 155, such as a client device in an examination room of the provider or a physician's office, also all have access to the health record. Authorized client devices as part of the provider's office network are identified either by IP addresses or MAC ID's which have been provided earlier This allows a physician for example to review the patient's health record on a client device 155 in the exam room while seeing the patient. Optionally, the physician adds information to the health record, such as a new diagnosis, from that same client device 155 in the exam room or later at yet another client device 155 on the network. In other embodiments, the information from the visit to the physician is determined from the insurance claim information provided by the physician to the insurance company. This allows for the health record to be updated without the physician having to do so manually.

Access—both to the basic insurance information and the health record of the patient—times out after a pre-determined amount of time. To access any information about the patient again, the authentication procedure must be re-initiated. The health record system 100 monitors 234 the amount of elapsed time either since the patient authenticated or since the last activity was initiated related to that patient at the client device 155 and closes 236 the authenticated session after the pre-determined amount of time. The pre-determined amount of time can be 15 min, 30 min or an hour or any other pre-determined amount of time.

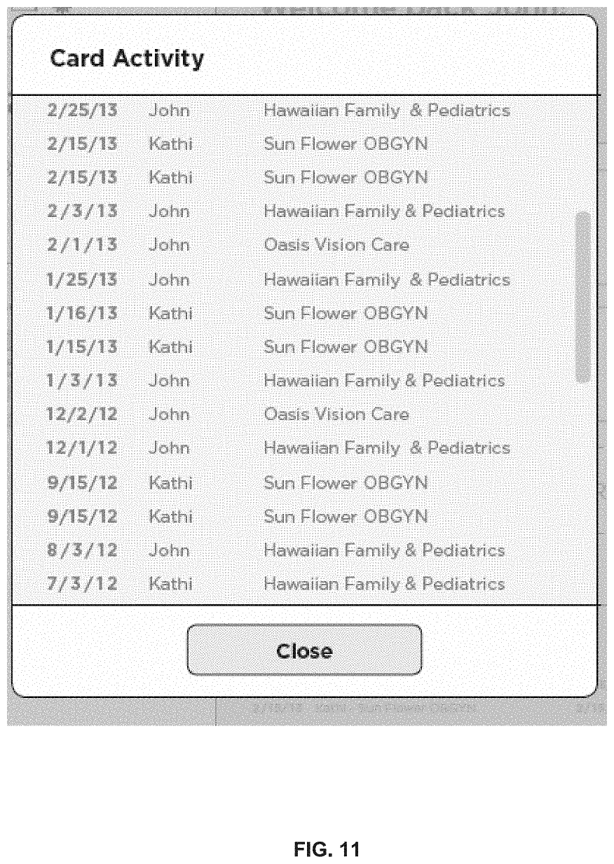

Each time a patient's health record is accessed, the access is logged in the patient's profile. A patient can access his or her health record and patient profile via a client device 155 and entering a password at a user interface. Upon accessing the health record, the user can add additional information. The additional information includes the type of information frequently requested manually when a patient arrives at a doctor's office such as allergies, past hospitalizations, etc. The patient can also view all accesses to the patient's health record by providers. FIG. 11 demonstrates such a list of accesses. In this example, the card provides access to health records for John and his wife Kathi. The list shows every time each record was accessed by date and provider.

### Process Overview—Linking of Profiles and Managing Consents

As shown in FIG. 8, and discussed above, a single card may provide access to health records for more than one patient. Each card is issued to a single patient and together with the PIN, provides access to the health record for that patient. Each patient then has an option to provide consent for their health record to be linked to and accessible via another patient's card. Once a second patient's health record is accessible via a first patient's card, that first patient has the ability to provide consent to access that second patient's health record. Thus when the second patient provides consent for his or her health record to be accessed via the first patient's health card, the second patient is providing consent for the first patient to consent to access to the second patient's health record.

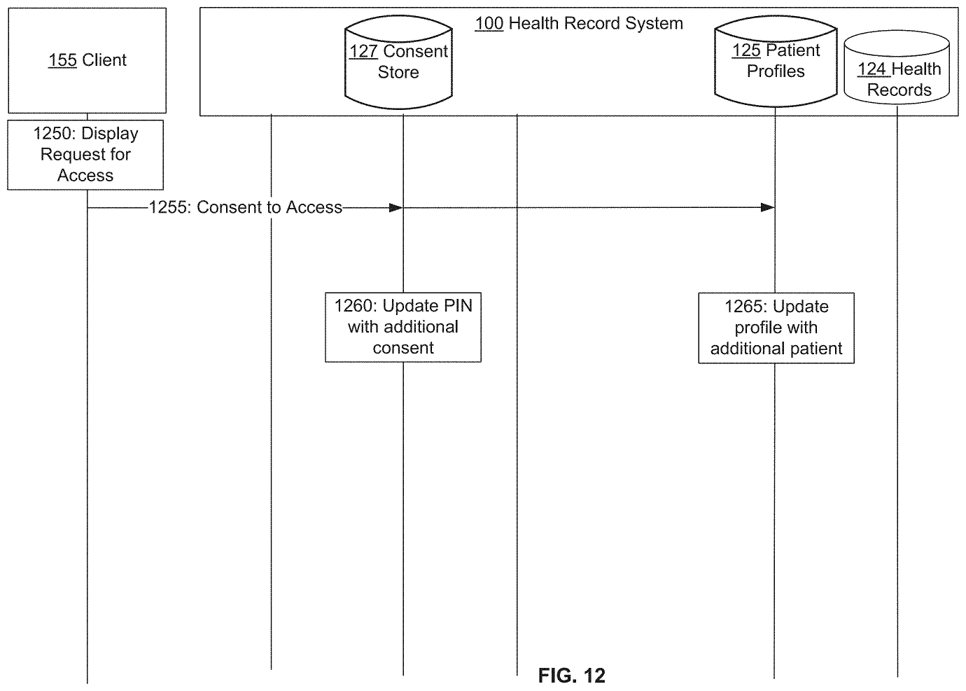

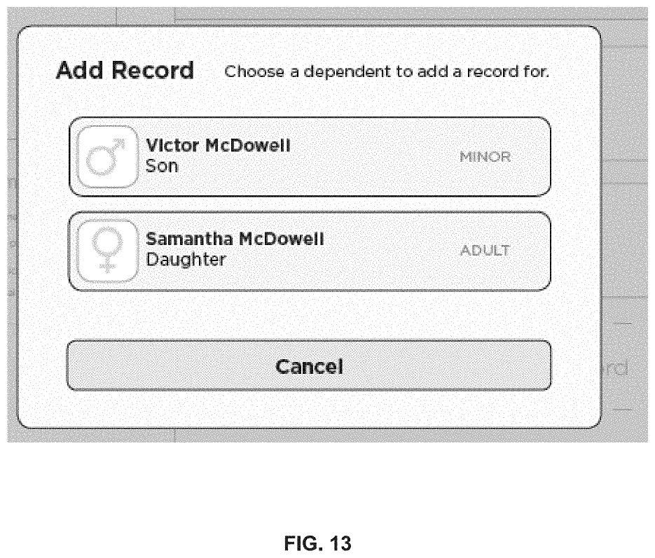

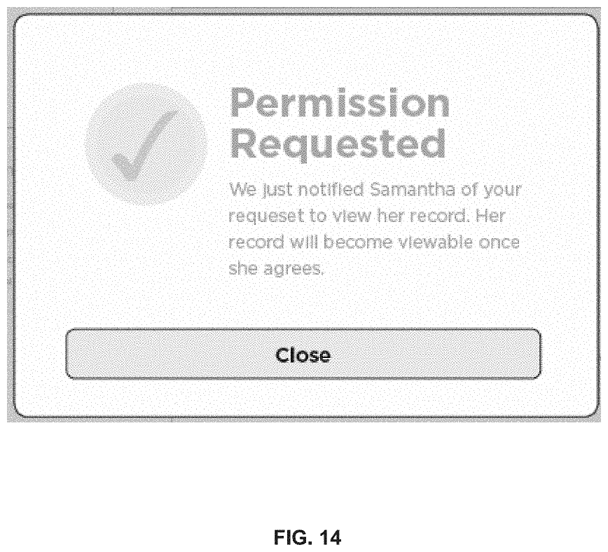

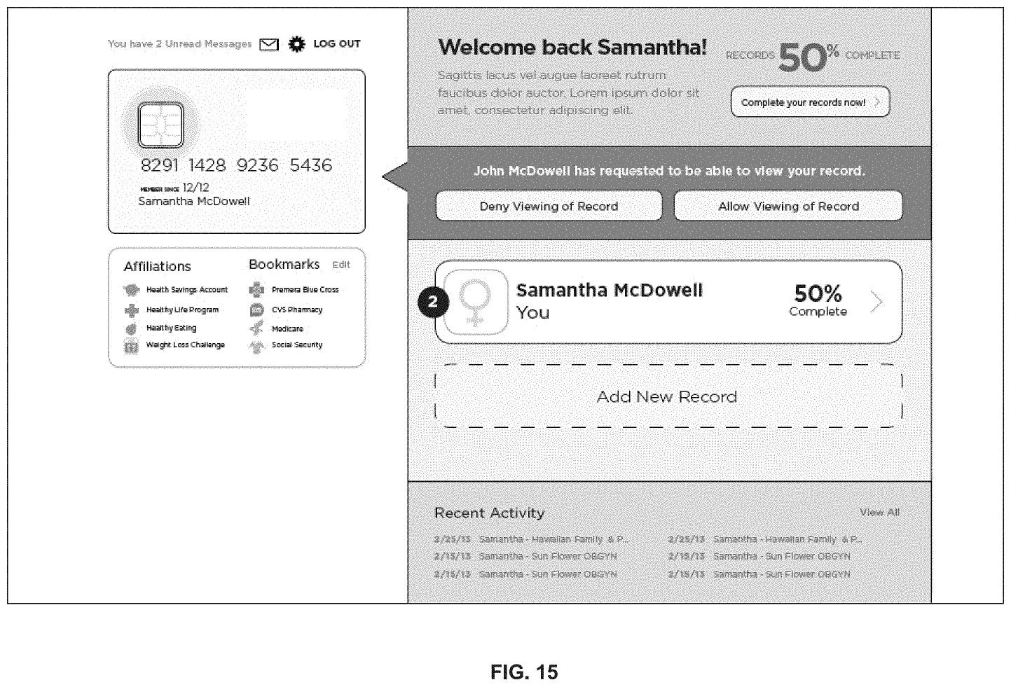

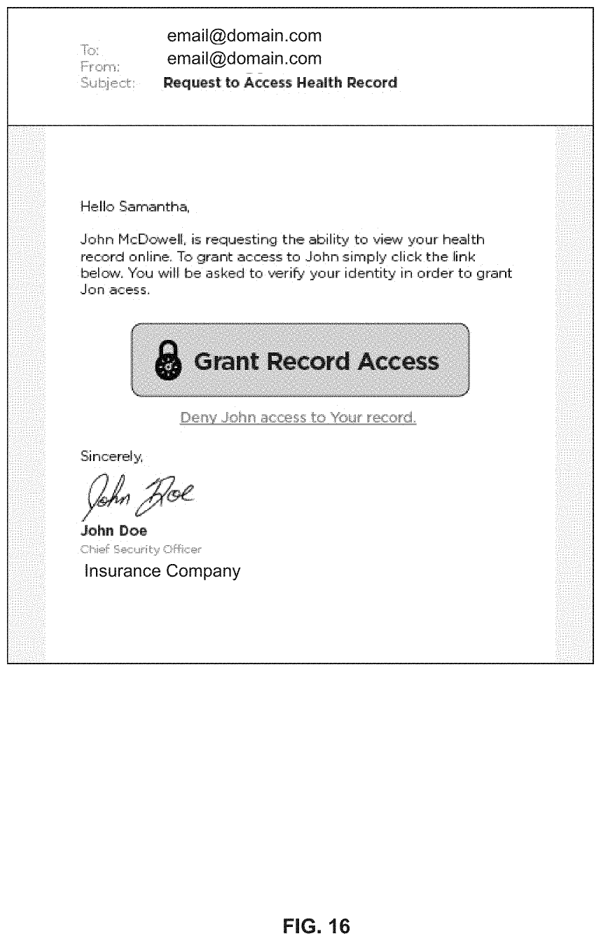

FIGS. 12-16 illustrate one embodiment of linking health records and allowing a first user to grant access to a second patient's medical records. FIG. 12 illustrates a data flow chart of providing a first user consent to allow access to a second user's health record. In FIG. 13, user John would like to enable access to health records for his dependents to his account. The user interface displayed to John at his client 155 lists the patients who are John's dependents listed on John's insurance—his son, Victor and his daughter Samantha. Victor is a minor and thus access to Victor's health record is added automatically to John's account. Thus in the consent store 127, John's PIN is associated with consents for Victor's health record as well as John's own. John can view his own and Victor's health record on his own client device 155 but can also enable access to his own and Victor's health record by a healthcare provider. Samantha is an adult and thus must provide consent to have her health record added to her father's account and thus allow him to provide access to that health record to providers. FIG. 14 illustrates the updated user interface presented to John indicating that permission is being requested from Samantha to add her health record to John's account. Referring to FIG. 12, John's request to view Samantha's health records and enable him to provide access to her records is displayed 1250 to Samantha at a client 155 she is using. In one embodiment, as illustrated in FIG. 15, the request is displayed to Samantha when she logs into her health record. In another embodiment, Samantha receives an email such as for example shown in FIG. 16. She has the option to allow or deny the access. In some embodiments, an electronic signature is captured in addition to or instead of a click on the “Allow Viewing of Record” button in FIG. 15 or the “Grant Record Access” button in FIG. 16. The electronic signature and/or the click received granting access is stored in Samantha's profile as documentation of Samantha's consent to John's access to her health record. Samantha consents 1255 to John having access to her health records and that consent is transmitted to the consent store 127 and the patient profiles 125. The consent store 127 updates 1260 John's PIN to add the consent to access Samantha's health record as associated with John's PIN. Additionally the patient profile for John in patient profiles 125 is updated 1265 with a link to Samantha's profile. Thus when John initially authenticates to the health record system 100, the interface at the client 155 where John is authenticating displays that Samantha as a linked patient. If John selects her profile and enters his PIN, her health record will be displayed.

Samantha can revoke permission for her father to view her health record and provide access to it. When the revocation is received by the health record system 100, her prior stored consent in the consent store 127 as associated with John's PIN is removed and the link to her health record is removed from John's patient profile. Thus when John presents his card at a provider, Samantha's health record will no longer appear as an option for the provider to access with John's PIN.

Additionally, FIG. 15 shows that Samantha has the option to add another patient's health record to her card as well.

The present disclosure has been described in particular detail with respect to several possible embodiments. Those of skill in the art will appreciate that the disclosure may be practiced in other embodiments. First, the particular naming of the modules, capitalization of terms, the attributes, data structures, or any other programming or structural aspect is not mandatory or significant, and the mechanisms that implement the disclosure or its features may have different names, formats, or protocols. Further, the system and the individual modules may be implemented as either software code executed by the computer system, or as hardware elements with dedicated circuit logic, or a combination of hardware and software. Also, the particular division of functionality between the various system components described herein is merely exemplary, and not mandatory; functions performed by a single system module may instead be performed by multiple modules, and functions performed by multiple modules may instead performed by a single module.

Some portions of above description present the features of the present disclosure in terms of methods and symbolic representations of operations on information. These descriptions and representations are the means used by those skilled in the data processing arts to most effectively convey the substance of their work to others skilled in the art. These operations, while described functionally or logically, are understood to be implemented by computer programs. Furthermore, it has also proven convenient at times, to refer to these arrangements of operations as modules or by functional names, without loss of generality.

Unless specifically stated otherwise as apparent from the above discussion, it is appreciated that throughout the description, discussions utilizing terms such as “determining” or “displaying” or the like, refer to the action and processes of a computer system, or similar electronic computing device, that manipulates and transforms data represented as physical (electronic) quantities within the computer system memories or registers or other such information storage, transmission or display devices.

Certain aspects of the present disclosure include process steps and instructions described herein in the form of a method. It should be noted that the process steps and instructions of the present disclosure could be embodied in software, firmware or hardware, and when embodied in software, could be downloaded to reside on and be operated from different platforms used by real time network operating systems.

The present disclosure also relates to an apparatus for performing the operations herein. This apparatus may be specially constructed for the required purposes, or it may comprise a general-purpose computer selectively activated or reconfigured by a computer program stored on a computer readable medium that can be accessed by the computer. Such a computer program may be stored in a tangible non-transitory computer readable storage medium, such as, but is not limited to, any type of disk including floppy disks, optical disks, CD-ROMs, magnetic-optical disks, read-only memories (ROMs), random access memories (RAMs), EPROMs, EEPROMs, magnetic or optical cards, application specific integrated circuits (ASICs), or any type of media suitable for storing electronic instructions, and each coupled to a computer system bus. Furthermore, the computers referred to in the specification may include a single processor or may be architectures employing multiple processor designs for increased computing capability.

Various general-purpose systems may be used with programs in accordance with the teachings herein, or it may prove convenient to construct more specialized apparatus to perform the required method steps. The required structure for a variety of these systems will be apparent to those of skill in the art, along with equivalent variations. In addition, the present disclosure is not described with reference to any particular programming language. It is appreciated that a variety of programming languages may be used to implement the teachings of the present disclosure as described herein, and any references to specific languages are provided for disclosure of enablement and best mode of the present disclosure.

The present disclosure is well suited to a wide variety of computer network systems over numerous topologies. Within this field, the configuration and management of large networks comprise storage devices and computers that are communicatively coupled to dissimilar computers and storage devices over a network, such as the Internet, public networks, private networks, or other networks enabling communication between computing systems. Finally, it should be noted that the language used in the specification has been principally selected for readability and instructional purposes, and may not have been selected to delineate or circumscribe the inventive subject matter. Accordingly, the disclosure of the present disclosure is intended to be illustrative, but not limiting, of the scope of the disclosure, which is set forth in the following claims.

1. A computer-implemented method for providing access to a health record to a health care provider comprising:
receiving at a computer system from a client device a first user consent for a second user to access a health record associated with the first user, the health record stored at the computer system;
storing at the computer system the consent as associated with a first authentication information associated with the second user;
receiving at the computer system from the second user at the first authentication information and a second authentication information;
receiving a request at the computer system for the health record associated with the first user; and
responsive to the received request and the received first and second pieces of authenticating information, providing the health record associated with the first user to a second client device.

2. The method of claim 1 further comprising receiving a third authentication information from a first healthcare provider and wherein providing the health record associated with the first user comprises providing the health record associated with the first user to the healthcare provider.

3. The method of claim 2 further comprising receiving from the second user information identifying a second healthcare provider and providing the health record associated with the first user is further responsive to matching the first healthcare provider as the same as the second healthcare provider.

4. The method of claim 1 further comprising:
receiving from the first user a withdrawal of the consent for the second user to access a health record associated with the first user; and
updating the first authentication information associated with the second user to remove the stored consent.

5. The method of claim 1 further comprising updating a user profile for the second user identifying the second user as associated with the first user.

6. The method of claim 1 wherein the first and second authentication information is received from a mobile device.

7. The method of claim 1 further comprising:
measuring an amount of elapsed time starting when providing the health record associated with the first user to the second client device; and
responsive to the amount of elapsed time exceeding a threshold, stop providing the health record to the second client device.

8. A computer system for providing access to a health record to a health care provider comprising:
a processor; and
a non-transitory computer-readable storage medium storing computer code, the code when executed by the processor causes the processor to perform steps comprising:
receiving from a client device a first user consent for a second user to access a health record associated with the first user, the health record stored at the computer system;
storing the consent as associated with a first authentication information associated with the second user;
receiving from the second user at the first authentication information and a second authentication information;
receiving a request for the health record associated with the first user; and
responsive to the received request and the received first and second pieces of authenticating information, providing the health record associated with the first user to a second client device.

9. The system of claim 8 further comprising code when executed by the processor causes the processor to perform steps comprising receiving a third authentication information from a first healthcare provider and wherein providing the health record associated with the first user comprises providing the health record associated with the first user to the healthcare provider.

10. The system of claim 9 further comprising code when executed by the processor causes the processor to perform steps comprising receiving from the second user information identifying a second healthcare provider and providing the health record associated with the first user is further responsive to matching the first healthcare provider as the same as the second healthcare provider.

11. The system of claim 8 further comprising code when executed by the processor causes
the processor to perform steps comprising:
receiving from the first user a withdrawal of the consent for the second user to access a health record associated with the first user; and
updating the first authentication information associated with the second user to remove the stored consent.

12. The system of claim 8 further comprising code when executed by the processor causes the processor to perform steps comprising updating a user profile for the second user identifying the second user as associated with the first user.

13. The system of claim 8 wherein the first and second authentication information is received from a mobile device.

14. A computer program product comprising a non-transitory computer-readable storage medium containing executable computer program code for controlling a processor to perform steps comprising:
receiving from a client device a first user consent for a second user to access a health record associated with the first user, the health record stored at the computer system;
storing the consent as associated with a first authentication information associated with the second user;
receiving from the second user at the first authentication information and a second authentication information;
receiving a request for the health record associated with the first user; and
responsive to the received request and the received first and second pieces of authenticating information, providing the health record associated with the first user to a second client device.

15. The computer program product of claim 14 further comprising computer program code for controlling a processor to perform steps comprising receiving a third authentication information from a first healthcare provider and wherein providing the health record associated with the first user comprises providing the health record associated with the first user to the healthcare provider.

16. The computer program product of claim 15 further comprising computer program code for controlling a processor to perform steps comprising receiving from the second user information identifying a second healthcare provider and providing the health record associated with the first user is further responsive to matching the first healthcare provider as the same as the second healthcare provider.

17. The computer program product of claim 14 further comprising computer program code for controlling a processor to perform steps comprising:
receiving from the first user a withdrawal of the consent for the second user to access a health record associated with the first user; and
updating the first authentication information associated with the second user to remove the stored consent.

18. The computer program product of claim 14 further comprising computer program code for controlling a processor to perform steps comprising updating a user profile for the second user identifying the second user as associated with the first user.

19. The computer program product of claim 14 wherein the first and second authentication information is received from a mobile device.
